(19) United States
(12) Patent Application Publication
(10) Pub. No.: US 2023/0019053 A1
(43) Pub. Date: Jan. 19, 2023

(54) GENOTYPING VARIABLE NUMBER TANDEM REPEATS

(71) Applicant: Illumina, Inc., San Diego, CA (US)

(72) Inventors: Sai Chen, San Diego, CA (US); Egor Dolzhenko, San Diego, CA (US); Michael A. Eberle, San Diego, CA (US)

(21) Appl. No.: 17/839,075

(22) Filed: Jun. 13, 2022

Related U.S. Application Data
(60) Provisional application No. 63/210,294, filed on Jun. 14, 2021.

(51) Int. Cl. G16B 30/00; G16B 20/10; G16B 40/00

(52) U.S. Cl. CPC G16B 30/00; G16B 20/10; G16B 40/00

(57) ABSTRACT

Disclosed herein include systems, devices, and methods for determining a variable number tandem repeat (VNTR) status. Haplotypes of a VNTR can be determined using long sequence reads of reference samples aligned to the VNTR in a reference. Short reads of a test sample of a test subject can be aligned to the haplotypes determined using the long sequence reads to determine a VNTR status (e.g., one or more haplotypes or a genotype of the test subject) of the test subject based on the probability indications of the haplotypes.

### RELATED APPLICATIONS

The present application claims priority under 35 U.S.C. § 119(e) to U.S. Provisional Application No. 63/210,294, filed Jun. 14, 2021. The content of the related application is incorporated herein by reference in its entirety.

### REFERENCE TO SEQUENCE LISTING

The present application is being filed along with a Sequence Listing in electronic format. The Sequence Listing is provided as a file entitled Sequence Listing 47CX-311979-US, created May 29, 2022 which is 1 kilobyte in size. The information in the electronic format of the Sequence Listing is incorporated herein by reference in its entirety.

### BACKGROUND

### Field

This disclosure relates generally to the field of processing sequencing data, and more particularly to genotyping variable number tandem repeats.

### Background

Variable nucleotide tandem repeats (VNTRs) account for a significant proportion of between-genome variation. Accurate detection of VNTRs have long been complicated by the low-complexity nature of the region and the length of the repetitive sequences. The detection power of VNTRs in existing short read pipelines needs improvements.

### SUMMARY

Disclosed herein include methods of determining a variable number tandem repeat (VNTR) status, such as genotyping the VNTR. In some embodiments, a method of determining a VNTR status is under control of a processor (e.g., a hardware processor or a virtual processor) and comprises: receiving a plurality of long sequence reads generated from a plurality of first samples obtained from a plurality of first subjects. The method can comprise: determining a plurality of haplotypes of a VNTR using long sequence reads of the plurality of long sequence reads aligned to the VNTR in a reference (e.g., a reference human genome sequence, such as hg19 or hg38). The method can comprise: receiving a plurality of short sequence reads generated from a second sample obtained from a second subject. The method can comprise: for each of the plurality of haplotypes of the VNTR, realigning short sequence reads, of the plurality of short sequence reads aligned to the VNTR, to the haplotype to generate a realignment. The method can comprise: determining a probability indication of each of the plurality of haplotypes of the VNTR for the second subject using the realignment of the short sequence reads realigned to the haplotype. The method can comprise: determining a status of the VNTR of the second subject based on the probability indications of each of the plurality of haplotypes. In some embodiments, the method comprises: generating a user interface (UI) comprising a UI element representing or comprising the status of the VNTR.

In some embodiments, a haplotype of the plurality of haplotypes of the VNTR is associated with a disease (e.g., bipolar disorder or monogenic diabetes). In some embodiments, determining the plurality of haplotypes of the VNTR comprises building or creating a database comprising the plurality of haplotypes of the VNTR. In some embodiments, determining the plurality of haplotypes of the VNTR comprises, for each of the plurality of first samples: extracting the long sequence reads of the plurality of long sequence reads of the first sample aligned to the VNTR in the reference. Determining the plurality of haplotypes of the VNTR can comprise: realigning the long sequence reads extracted to a left flanking region and a right flanking region of the VNTR to determine aligned long sequence reads. Determining the plurality of haplotypes of the VNTR can comprise: determining a haplotype of the plurality of haplotypes based on the aligned long sequence reads each with an alignment score above an alignment threshold. At least one of the long sequence reads of the plurality of long sequence reads of the first sample is aligned to the VNTR and/or realigned to the left flanking region and the right flanking region span the VNTR. In some embodiments, determining the haplotype of the plurality of haplotypes of the VNTR comprises: trimming sequences, of the aligned long sequence reads each with the alignment score above the alignment threshold, aligned to the left flanking region and the right flanking region to generate trimmed long sequence reads. Determining the haplotype of the plurality of haplotypes of the VNTR can comprise: determining the haplotype of the plurality of haplotypes based on the trimmed long sequence reads.

In some embodiments, the first sample is homozygous for the VNTR. Determining the haplotype of the plurality of haplotypes can comprise: determining only one haplotype of the plurality of haplotypes based on the trimmed long sequence reads. Determining the only one haplotype can comprise: determining the only one haplotype can comprise: clustering the trimmed long sequence reads into only one cluster. Clustering the trimmed long sequence reads into the only one cluster can comprise: clustering the trimmed long sequence reads into the only one cluster based on lengths of the trimmed long sequence reads. The clustering can comprise k-means clustering. Determining the only one haplotype can comprise: determining the only one haplotype based on the trimmed long sequence reads.

In some embodiments, the first sample is heterozygous for the VNTR. Determining the haplotype of the plurality of haplotypes can comprise: determining two haplotypes of the plurality of haplotypes of the VNTR based on the trimmed long sequence reads. Determining the two haplotypes can comprise: clustering the trimmed long sequence reads into two clusters. Clustering the trimmed long sequence reads into the two clusters can comprise: clustering the trimmed long sequence reads into the two clusters based on lengths of the trimmed long sequence reads. The clustering can comprise k-means clustering. Determining the two haplotypes can comprise: determining a first haplotype of the two haplotypes based on the trimmed long sequence reads in a first cluster of the two clusters. Determining the two haplotypes can comprise: determining a second haplotype of the two haplotypes based on the trimmed long sequence reads in a second cluster of the two clusters. In some embodiments, the trimmed long sequence reads comprise a first plurality of trimmed long sequence reads and a second plurality of trimmed long sequence reads with different lengths. The different lengths differ by at least 5,000 base pairs. The first cluster can comprise all, substantially all, or a majority of the first plurality of trimmed long sequence reads. The second cluster can comprise all, substantially all, or majority of the second plurality of trimmed long sequence reads.

In some embodiments, determining the haplotype of the plurality of haplotypes of the VNTR comprises: determining a consensus sequence of the trimmed long sequence reads. In some embodiments, determining the consensus sequence of the trimmed long sequence reads comprises, for each position of each of the trimmed long sequence reads with a base that is not the most frequent base amongst the trimmed long sequence reads at the position: modifying the trimmed long sequence read at the position using each of a plurality of operations (a delete operation, an insert operation, and a replace operation) independently and determining a sum of distances (e.g., edit distances) between (i) a modified trimmed long sequence read resulting from the operation on the trimmed long sequence read at the base and (ii) the trimmed long sequence reads other than the trimmed long sequence read being modified. Determining the consensus sequence of the trimmed long sequence reads can comprise: modifying the trimmed long sequence at the base using the operation of the plurality of operations resulting in the smallest sum of distances (e.g., edit distances) amongst the plurality of operations or replacing the trimmed long sequence read with the modified trimmed long sequence read corresponding to the smallest sum of distances (e.g., edit distances).

In some embodiments, determining the consensus sequence of the trimmed long sequence reads comprises, for each corresponding position of the trimmed long sequence reads: determining a most frequent base amongst bases of the trimmed long sequence reads at the position. Determining the consensus sequence of the trimmed long sequence reads can comprise, for each of the trimmed long sequence reads with bases at the position that are not the most frequent base at the position: determining a sum of distances (e.g., edit distances) between (i) a modified trimmed long sequence read resulting from each of a plurality of operations (e.g., a delete operation, an insert operation, and a replace operation) independently on the trimmed long sequence read and (ii) the trimmed long sequence reads other than the trimmed long sequence read being modified. Determining the consensus sequence of the trimmed long sequence reads can comprise: determining the smallest sum of distances (e.g., edit distances) amongst the sums of distances (e.g., edit distances). Determining the consensus sequence of the trimmed long sequence reads can comprise: modifying the trimmed long sequence read at the base with the operation resulting in the smallest sum of distances (e.g., edit distances) or replacing the trimmed long sequence read with the modified trimmed long sequence read corresponding to the smallest sum of distances (e.g., edit distances). In some embodiments, the plurality of operations comprises: deleting the base of the trimmed long sequence at the position. The plurality of operations can comprise: inserting the most frequent base at the position into the trimmed long sequence at the position. The plurality of operations can comprise: replacing the base of the trimmed long sequence at the position with the most frequent base at the position.

In some embodiments, qualities of the long sequence reads of the plurality of long sequence reads aligned to the VNTR in the reference satisfy quality criteria. Qualities of the plurality of haplotypes can satisfy quality criteria.

In some embodiments, the status of the VNTR comprises a haplotype status of the VNTR. The haplotype status can comprise a haplotype, a length of the haplotype, and/or a confidence interval of the length of the haplotype. The status of the VNTR can comprise a genotype status of the VNTR. The genotype status can comprise a genotype, lengths of the haplotypes of the genotype, and/or a confidence interval of the length of each of the haplotypes of the genotype. The confidence interval can comprise a shortest length of the haplotype and a longest length of the haplotype.

In some embodiments, determining the status of the VNTR of the second subject comprises: determining two or more haplotypes of the plurality of haplotypes with the probability indications satisfy a probability criterium. Determining the status of the VNTR of the second subject can comprise: determining lengths of the two or more haplotypes determined. The shortest length of the haplotype can be the shortest length of the lengths of the two or more haplotypes determined. The longest length of the haplotypes can be the longest length of the lengths of the two or more haplotypes determined. In some embodiments, an accuracy of the status of the VNTR is at least 60%.

In some embodiments, the probability indication of each of the plurality of haplotypes of the VNTR comprises a probability of each of the plurality of haplotypes of the VNTR. The probability criterium can comprise a probability threshold.

In some embodiments, the plurality of long sequence reads comprises sequence reads that are about 10,000 base pairs to about 20,000 base pairs in length each. The plurality of long sequence reads can be generated by targeted sequencing or whole genome sequencing (WGS). The WGS can be clinical WGS (cWGS). The plurality of first subjects can comprises human subject.

In some embodiments, the plurality of short sequence reads can comprise sequence reads that are about 100 base pairs to about 1000 base pairs in length each. The plurality of short sequence reads can comprise paired-end sequence reads. The plurality of short sequence reads can comprise single-end sequence reads. The plurality of short sequence reads can be generated by targeted sequencing or whole genome sequencing (WGS). The WGS can be clinical WGS (cWGS). The second subject can comprise a human subject.

In some embodiments, the plurality of first subjects comprises the second subject. The plurality of first samples can comprise the second sample. In some embodiments, the plurality of first samples and/or the second sample comprises cells, cell-free DNA, cell-free fetal DNA, amniotic fluid, a blood sample, a biopsy sample, or a combination thereof. The plurality of first samples can comprise at least 50 samples.

In some embodiments, each haplotype of the plurality of haplotypes of the VNTR comprises a plurality of copies of a repeat unit. The repeat unit can be more than six base pairs in length. The number of the plurality of copies can be at least three. In some embodiments, sequences of two copies of the plurality of copies of the repeat unit of a haplotype of the plurality of haplotypes are different at one or more differentiating positions. The sequences of the two copies of the plurality of copies of the repeat unit of a haplotype have at least 80% sequence identify. Sequences of two copies of the plurality of copies of the repeat unit of a haplotype of the plurality of haplotypes can be identical. In some embodiments, two haplotypes of the plurality of haplotypes of the VNTR comprise different numbers of copies of the repeat unit. In some embodiments, two haplotypes of the plurality of haplotypes of the VNTR comprise an identical number of copies of the repeat unit. In some embodiments, a sequence of a copy of the repeat unit of one of the two haplotypes and a sequence of a copy of the repeat unit of the other one of the two haplotypes are different at one or more differentiating positions. The sequences can have at least 80% sequence identity. A sequence of a copy of the repeat unit of one of the two haplotypes and a sequence of a copy of the repeat unit of the other one of the two haplotypes can be identical.

Disclosed herein include systems of determining a variable number tandem repeat (VNTR) status, such as genotyping the VNTR. In some embodiments, a system for determining a VNTR status comprises: non-transitory memory configured to store executable instructions and a plurality of haplotypes of a VNTR. The system can comprise: a processor (e.g., a hardware processor or a virtual processor) in communication with the non-transitory memory, the processor programmed by the executable instructions to perform: receiving a plurality of short sequence reads generated from a test sample obtained from a test subject. The processor can be programmed by the executable instructions to perform: for each of the plurality of haplotypes of the VNTR, realigning short sequence reads, of the plurality of short sequence reads aligned to the VNTR, to the haplotype to generate a realignment. The processor can be programmed by the executable instructions to perform: determining a probability of each of the plurality of haplotypes for the test subject using the realignment of the short sequence reads realigned to the haplotype. The processor can be programmed by the executable instructions to perform: determining a status of the VNTR of the test subject. In some embodiments, the processor is programmed by the executable instructions to perform: determining a user interface (UI) comprising a UI element representing or comprising the status of the VNTR.

In some embodiments, wherein a haplotype of the plurality of haplotypes of the VNTR is associated with a disease (e.g., bipolar disorder or monogenic diabetes). In some embodiments, the plurality of haplotypes of the VNTR is determined using long sequence reads of a plurality of long sequence reads aligned to the VNTR in a reference (e.g., reference human genome sequence, such as hg19 or hg38). In some embodiments, the plurality of long sequence reads can be generated from a plurality of reference samples obtained from a plurality of reference subjects. The plurality of haplotypes of the VNTR can be determined by: for each of the plurality of samples: extracting the long sequence reads of the plurality of long sequence reads of the test sample aligned to the VNTR in the reference. The plurality of haplotypes of the VNTR can be determined by: realigning the long sequence reads extracted to a left flanking region and a right flanking region of the VNTR to determine aligned long sequence reads. The plurality of haplotypes of the VNTR can be determined by: determining a haplotype of the plurality of haplotypes based on the aligned long sequence reads each with an alignment score above an alignment threshold. At least one of the long sequence reads of the plurality of long sequence reads of the test sample can be aligned to the VNTR. At least one of the long sequence reads of the plurality of long sequence reads of the test sample can be realigned to the left flanking region and the right flanking region span the VNTR. In some embodiments, the haplotype of the plurality of haplotypes of the VNTR is determined by: trimming sequences, of the aligned long sequence reads each with the alignment score above the alignment threshold, aligned to the left flanking region and the right flanking region to generate trimmed long sequence reads. The haplotype of the plurality of haplotypes of the VNTR can be determined by: determining the haplotype of the plurality of haplotypes based on the trimmed long sequence reads.

In some embodiments, the reference sample is homozygous for the VNTR. The haplotype of the plurality of haplotypes of the VNTR can be determined to comprise one haplotype of the plurality of haplotypes based on the trimmed long sequence reads. The only one haplotype can be determined by: clustering the trimmed long sequence reads into only one cluster. Clustering the trimmed long sequence reads into the only one cluster can comprise: clustering the trimmed long sequence reads into the only one cluster based on lengths of the trimmed long sequence reads. The clustering can comprise k-means clustering. The only one haplotype can be determined by: determining the only one haplotype based on the trimmed long sequence reads.

In some embodiments, the reference sample is heterozygous for the VNTR. The haplotype of the plurality of haplotypes of the VNTR can be determined to comprise two haplotypes of the plurality of haplotypes based on the trimmed long sequence reads. The two haplotypes can be determined by: clustering the trimmed long sequence reads into two clusters. Clustering the trimmed long sequence reads into the two clusters can comprise: clustering the trimmed long sequence reads into the two clusters based on lengths of the trimmed long sequence reads. The clustering can comprise k-means clustering. The two haplotypes can be determined by: determining a first haplotype of the two haplotypes based on the trimmed long sequence reads in a first cluster of the two clusters. The two haplotypes can be determined by: determining a second haplotype of the two haplotypes based on the trimmed long sequence reads in a second cluster of the two clusters. In some embodiments, the trimmed long sequence reads comprises a first plurality of trimmed long sequence reads and a second plurality of trimmed long sequence reads with different lengths. The different lengths differ by at least 5,000 base pairs. The first cluster can comprise all, substantially all, or a majority of the first plurality of trimmed long sequence reads. The second cluster can comprise all, substantially all, or a majority of the second plurality of trimmed long sequence reads.

In some embodiments, to determine the haplotype of the plurality of haplotypes of the VNTR, a consensus sequence of the trimmed long sequence reads is determined. In some embodiments, the consensus sequence of the trimmed long sequence reads is determined by: for each position of each of the trimmed long sequence reads with a base that is not the most frequent base amongst the trimmed long sequence reads at the position: modifying the trimmed long sequence read at the position using each of a plurality of operations (e.g., a delete operation, an insert operation, and a replace operation) and determining a sum of edit distances between (i) a modified trimmed long sequence read resulting from the operation on the trimmed long sequence read at the base and (ii) the trimmed long sequence reads other than the trimmed long sequence read being modified; and modifying the trimmed long sequence at the base using the operation of the plurality of operations resulting in the smallest sum of edit distances amongst the plurality of operations or replacing the trimmed long sequence read with the modified trimmed long sequence read corresponding to the smallest sum of edit distances.

In some embodiments, the consensus sequence of the trimmed long sequence reads is determined by: for each corresponding position of the trimmed long sequence reads: determining a most frequent base amongst bases of the trimmed long sequence reads at the position; for each of the trimmed long sequence reads with bases at the position that are not the most frequent base at the position: for each of a plurality of operations (e.g., a delete operation, an insert operation, and a replace operation), determining a sum of edit distances between (i) a modified trimmed long sequence read resulting from the operation on the trimmed long sequence read and (ii) the trimmed long sequence reads other than the trimmed long sequence read being modified; determining the smallest sum of edit distances amongst the sums of edit distances; and modifying the trimmed long sequence read at the base with the operation resulting in the smallest sum of edit distances or replacing the trimmed long sequence read with the modified trimmed long sequence read corresponding to the smallest sum of edit distances. In some embodiments, the plurality of operations comprises: deleting the base of the trimmed long sequence at the position, inserting the most frequent base at the position into the trimmed long sequence at the position, and replacing the base of the trimmed long sequence at the position with the most frequent base at the position.

In some embodiments, qualities of the long sequence reads of the plurality of long sequence reads aligned to the VNTR in the reference satisfy quality criteria. Qualities of the plurality of haplotypes can satisfy quality criteria.

In some embodiments, the status of the VNTR comprises a haplotype status of the VNTR. The haplotype status can comprise a haplotype, a length of the haplotype, and/or a confidence interval of the length of the haplotype. The status of the VNTR can comprise a genotype status of the VNTR, The genotype status can comprise a genotype, lengths of the haplotypes of the genotype, and/or a confidence interval of the length of each of the haplotypes of the genotype. The confidence interval can comprise a shortest length of the haplotype and a longest length of the haplotype.

In some embodiments, determining the haplotype status of the VNTR of the test subject comprises: determining two or more haplotypes of the plurality of haplotypes with the probability indications satisfy a probability criterium. Determining the haplotype status of the VNTR of the test subject can comprise: determining lengths of the two or more haplotypes determined. The shortest length of the haplotype can be the shortest length of the lengths of the two or more haplotypes determined. The longest length of the haplotypes can be the longest length of the lengths of the two or more haplotypes determined. In some embodiments, an accuracy of the haplotype status is at least 60%.

In some embodiments, the probability indication of each of the plurality of haplotypes of the VNTR comprises a probability of each of the plurality of haplotypes of the VNTR. The probability criterium can comprise a probability threshold.

In some embodiments, the plurality of long sequence reads comprises sequence reads that are about 10,000 base pairs to about 20,000 base pairs in length each. The plurality of long sequence reads can be generated by targeted sequencing or whole genome sequencing (WGS). The WGS can be clinical WGS (cWGS). The plurality of reference subjects can comprise human subject.

In some embodiments, the plurality of short sequence reads comprises sequence reads that are about 100 base pairs to about 1000 base pairs in length each. The plurality of short sequence reads can comprise paired-end sequence reads. The plurality of short sequence reads can comprise single-end sequence reads. The plurality of short sequence reads can be generated by targeted sequencing or whole genome sequencing (WGS). The WGS can be clinical WGS (cWGS). The test subject can comprise a human subject. A first sample can comprise cells, cell-free DNA, cell-free fetal DNA, amniotic fluid, a blood sample, a biopsy sample, or a combination thereof. A first subject can be a human subject.

In some embodiments, the plurality of reference subjects comprises the test subject. The plurality of reference samples can comprise the test sample. In some embodiments, the plurality of reference samples and/or the test sample comprises cells, cell-free DNA, cell-free fetal DNA, amniotic fluid, a blood sample, a biopsy sample, or a combination thereof. The plurality of reference samples can comprise at least 50 samples.

In some embodiments, each haplotype of the plurality of haplotypes of the VNTR comprises a plurality of copies of a repeat unit. The repeat unit can be more than six base pairs in length. The number of the plurality of copies can be at least three. In some embodiments, sequences of two copies of the plurality of copies of the repeat unit of a haplotype of the plurality of haplotypes are different at one or more differentiating positions. The sequences of the two copies of the plurality of copies of the repeat unit of a haplotype have at least 80% sequence identify. Sequences of two copies of the plurality of copies of the repeat unit of a haplotype of the plurality of haplotypes can be identical. In some embodiments, two haplotypes of the plurality of haplotypes of the VNTR comprise different numbers of copies of the repeat unit. In some embodiments, two haplotypes of the plurality of haplotypes of the VNTR comprise an identical number of copies of the repeat unit. In some embodiments, a sequence of a copy of the repeat unit of one of the two haplotypes and a sequence of a copy of the repeat unit of the other one of the two haplotypes are different at one or more differentiating positions. The sequences can have at least 80% sequence identity. A sequence of a copy of the repeat unit of one of the two haplotypes and a sequence of a copy of the repeat unit of the other one of the two haplotypes can be identical.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 shows a non-limiting exemplary illustration of a VNTR in a reference sequence and in five samples.

FIG. 2 shows a non-limiting exemplary schematic illustration of building a VNTR database from long reads.

FIGS. 3A-3B show a non-limiting exemplary schematic illustration of generating a haplotype from multiple long reads.

FIG. 4 shows a non-limiting exemplary schematic illustration of genotype VNTRs on short reads.

FIG. 5 is a flow diagram showing an exemplary method of determining VNTR status (e.g., VNTR haplotypes or genotypes).

FIG. 6 is a block diagram of an illustrative computing system configured to implement determining VNTR status (e.g., VNTR haplotypes or genotypes).

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods of determining a variable number tandem repeat (VNTR) status, such as genotyping the VNTR. In some embodiments, a method of determining a VNTR status is under control of a processor (e.g., a hardware processor or a virtual processor) and comprises: receiving a plurality of long sequence reads generated from a plurality of first samples obtained from a plurality of first subjects. The method can comprise: determining a plurality of haplotypes of a VNTR using long sequence reads of the plurality of long sequence reads aligned to the VNTR in a reference (e.g., a reference human genome sequence, such as hg19 or hg38). The method can comprise: receiving a plurality of short sequence reads generated from a second sample obtained from a second subject. The method can comprise: for each of the plurality of haplotypes of the VNTR, realigning short sequence reads, of the plurality of short sequence reads aligned to the VNTR, to the haplotype to generate a realignment. The method can comprise: determining a probability indication of each of the plurality of haplotypes of the VNTR for the second subject using the realignment of the short sequence reads realigned to the haplotype. The method can comprise: determining a status of the VNTR of the second subject based on the probability indications of each of the plurality of haplotypes. In some embodiments, the method comprises: generating a user interface (UI) comprising a UI element representing or comprising the status of the VNTR.

Disclosed herein include systems of determining a variable number tandem repeat (VNTR) status, such as genotyping the VNTR. In some embodiments, a system for determining a VNTR status comprises: non-transitory memory configured to store executable instructions and a plurality of haplotypes of a VNTR. The system can comprise: a processor (e.g., a hardware processor or a virtual processor) in communication with the non-transitory memory, the processor programmed by the executable instructions to perform: receiving a plurality of short sequence reads generated from a test sample obtained from a test subject. The processor can be programmed by the executable instructions to perform: for each of the plurality of haplotypes of the VNTR, realigning short sequence reads, of the plurality of short sequence reads aligned to the VNTR, to the haplotype to generate a realignment. The processor can be programmed by the executable instructions to perform: determining a probability of each of the plurality of haplotypes for the test subject using the realignment of the short sequence reads realigned to the haplotype. The processor can be programmed by the executable instructions to perform: determining a status of the VNTR of the test subject. In some embodiments, the processor is programmed by the executable instructions to perform: determining a user interface (UI) comprising a UI element representing or comprising the status of the VNTR.

### Genotyping Variable Number Tandem Repeats

Disclosed herein include a genotyper that significantly improves variable number tandem repeats (VNTR) genotyping performance on short read sequencing data (e.g., sequencing data generated by sequencing methods such as sequencing-by-synthesis). For example, the improvement was made by utilizing a pre-constructed VNTR database. As another example, the improvement was made by optimizing current genotyping methods on low-complexity regions. The present disclosure also provides a workflow that is capable of constructing a population VNTR database from, for example, Pacific Biosciences of California, Inc. (PacBio, Menlo Park, Calif.) HiFi data.

A VNTR can be a repeat sequence where the repeat is greater than 6 base pairs (bps) in length and the repeat region is greater than 80% pure (fewer than 20% mismatches for an exact repeat). Structural variations (SVs) in VNTRs include insertion/deletion of the repetitive sequences. Variations can be highly population-specific. Some VNTRs are known to cause to genetic diseases, such as bipolar disorder and monogenic diabetes. VNTRs account for significant proportion of per-sample variation. Around half of all SVs (greater than 10k) per individual can be classified as VNTRs. On average one person has about 2.2 mega base pairs (Mbps) of deleted sequence and about 5.7 Mbps of inserted sequence in VNTRs.

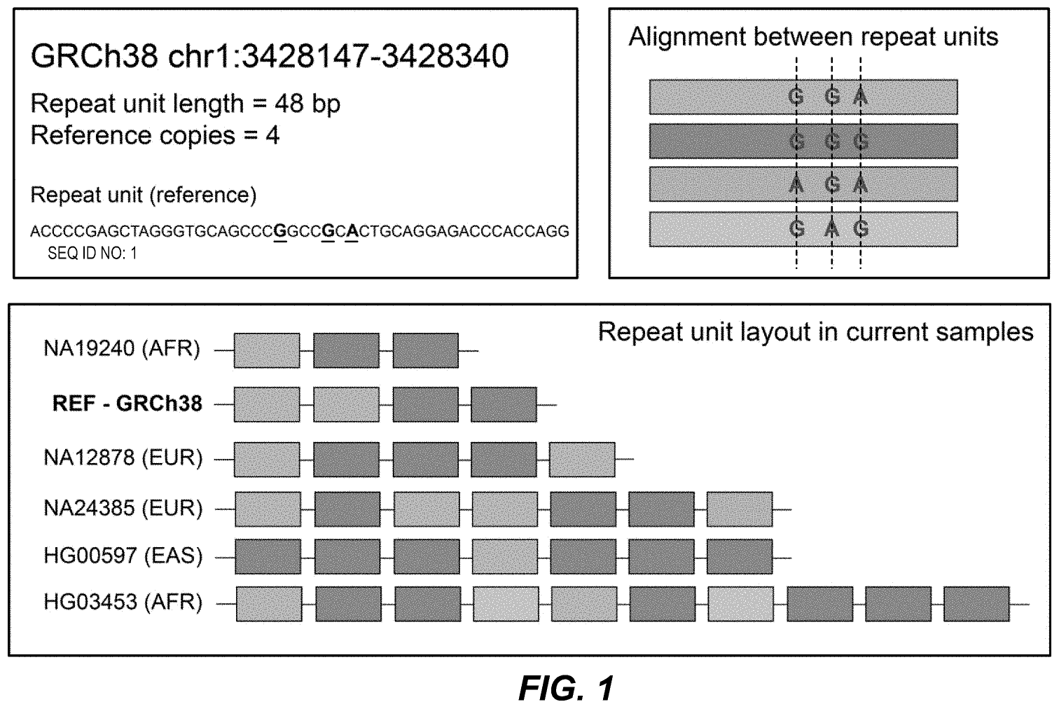

FIG. 1 shows a non-limiting exemplary illustration of a VNTR in a reference sequence and five samples. The VNTR in the reference human genome GRCh38 is at chr1:3428147-3428340 (FIG. 1, top left panel). The repeat unit has a length of 48 bps. The reference sequence of the repeat unit is ACCCCGAGCTAGGGTGCAGCCCGGCCGCACTGCAGGAGACCCACCAGG (SEQ ID NO: 1) in GRCh38. Different copies of the repeat unit in the VNTR (within a haplotype or across haplotypes) can vary, in particular at the three bases bolded and underlined. The three bases can be G, G, and A, respectively, in a first type or sequence of the repeat unit; G, G, and G, respectively, in a second type or sequence of the repeat unit; A, G, and A, respectively, in a third type or sequence of the repeat unit; and G, A, and G, respectively, in a fourth type or sequence of the repeat unit (FIG. 1, top right panel). The VNTR includes four copies of the repeat unit in GRCh38 (FIG. 1, bottom panel). The four copies include two copies of the first type followed by two copies of the second type (FIG. 1, bottom panel). The five samples included three, five, seven, seven, and ten copies of the repeat unit respectively. For sample NA19240 of a subject who is African, the VNTR included one copy of the first type followed by two copies of the second type. For sample NA12878 of a subject who is European), the VNTR included one copy of the first type, three copies of the second type, and one copy of the first type. For sample NA24385 of a subject who is European, the VNTR included one copy of the first type, one copy of the second type, two copies of the first type, two copies of the second type, and one copy of the third type. For sample HG00597 of a subject who is Easter Asian, the VNTR included three copies of the second type, one copy of the first type, and three copies of the second type. For sample HG03453 of a subject who is African, the VNTR included one copy of the first type, two copies of the second type, one copy of the fourth type, one copy of the first type, one copy of the second type, one copy of the fourth type, and three copies of the second type.

VNTR genotyping is missing in short-read pipelines. Short reads often cannot cover the full length of most VNTRs. Short reads are also referred to herein as short sequence reads. Around 29% of the VNTRs have additional repeats with total length greater than or equal to 150 bps in one individual. Due to the repetitive nature of VNTRs, correctly rebuilding VNTRs' haplotypes from short reads is extremely difficult. VNTR detection power is extremely low in short-read pipelines. For example, DRAGEN v3.4 detection power for VNTRs is less than 20%.

Disclosed herein is a VNTR genotype implementing a VNTR genotyping method which includes one or more of the following. First, the method can include (or the VNTR genotyper can perform) building a database of common VNTR haplotypes in the population. Long reads (e.g., PacBio HiFi reads), which can be highly accurate, can be used to build a database of common VNTR haplotypes. Long reads are also referred to herein as long sequence reads. Second, the method can include extracting short reads from the target VNTR region generated by sequencing methods including sequencing-by-synthesis, such as short reads generated using a sequencing instrument from Illumina, Inc. (San Diego, Calif.). The these extracted short reads can be realigned to each haplotype sequence in the database. Third, the method can include deriving the most likely VNTR haplotypes (and thus genotype) from the realignments. VNTRs usually have differences between repeat units in different haplotypes. The differences between the repeat units can be referred to herein as the differentiating bases. The most likely haplotypes (and thus genotype) can be determined from these differentiating positions (See FIG. 1, top right panel for example differentiating bases within a haplotype and across haplotypes of a VNTR).

The method can include building a VNTR database from long reads (e.g., PacBio HiFi reads), which can be highly accurate. PacBio HiFi reads are long enough (15 kb on average) to span the full length of most VNTRs. Long read sequencing is limited by DNA input and cost and cannot be performed in a large scale. However, as described herein, sequencing some samples (e.g., a few hundred samples) to build the database is possible.

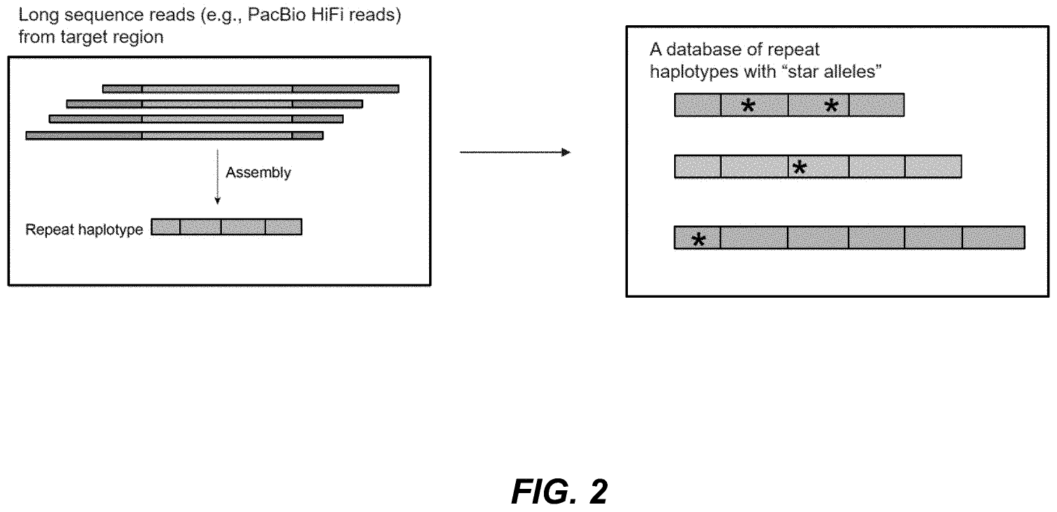

FIG. 2 shows an example of building a VNTR database from long reads. For each sample, long reads (e.g., PacBio HiFi reads) can be extracted from the target VNTR region. Reads can be aligned to the left and right flanking region of the VNTR Reads that have good alignments to flanking regions on both sides can be kept. The flanking regions can be trimmed off the reads. Whether the trimmed reads are from one haplotype or two haplotypes can be differentiated. For example, if reads can be clustered (e.g., k-means clustered) into two clusters, the sample is heterozygous. Otherwise the sample is homozygous. The haplotype(s) can be assembled from differentiated reads. For example, if the reads can be clustered into two clusters, the reads in each cluster can be assembled into a haplotype. The reads in the two clusters can be assembled into two haplotypes. If the reads cannot be clustered into two clusters, the reads can be assembled into a haplotype. The resulting database of repeat haplotypes can include “star alleles.” For example, haplotypes in the database can include differentiating bases that can be used to differentiate the haplotypes. Repeat units in the haplotypes can include differentiating bases that can be used to differentiate the haplotypes. The three haplotypes shown in FIG. 2 have four, five, and six copies of the repeat units respectively.

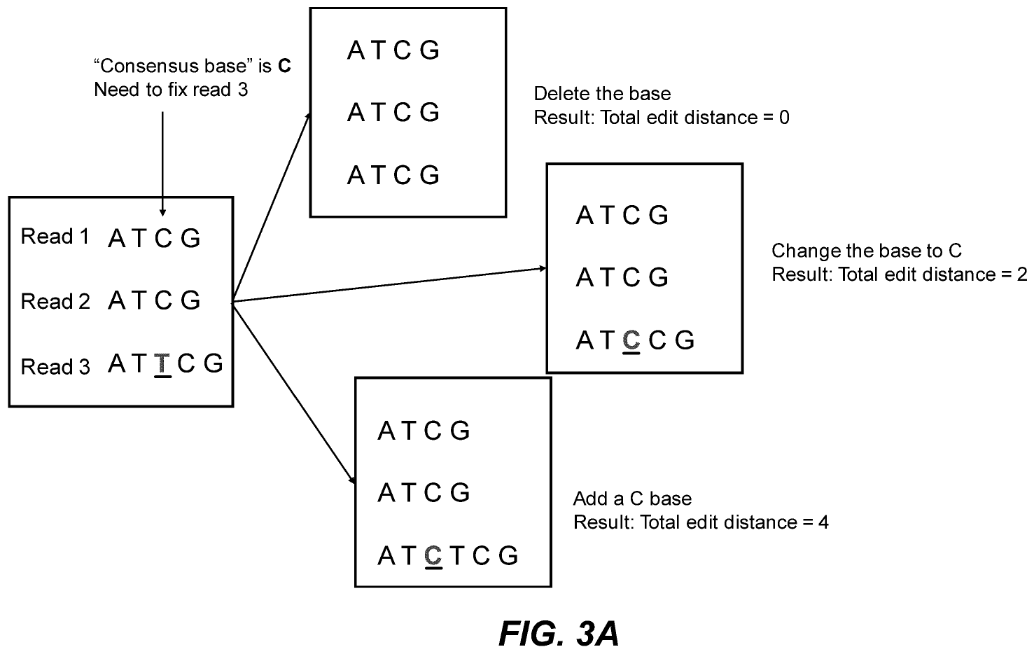

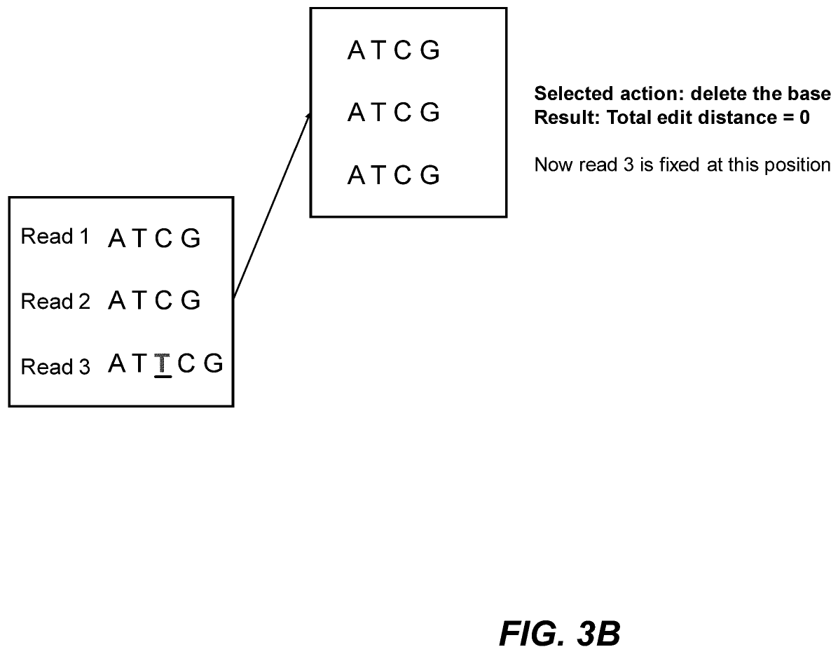

With a low sequencing error rate (e.g., less than 1%), observing different bases in each position should be rare. Accordingly, the method illustrated with reference to FIGS. 3A-3B can be used to correct sequencing errors and assemble the haplotype. For each position, label the base with the highest fraction (most common) amongst these reads (e.g., trimmed reads) as the “consensus base” (also referred to herein as the “truth base”). For each read (e.g., trimmed read) with a different base from the “consensus base,” perform the following three actions (or operations) independently: delete a base, add the “consensus base,” or change the base to the “consensus base.” The distance (e.g., edit distance) between the modified read (e.g., the read modified from the trimmed read) generated by each action (or operation) and each of the other reads (e.g., trimmed reads) can be calculated, and these distances can be summed. The read can be modified with the action (or operation) that has the smallest sum of distances. The sum of the distances for an action and the sum of the distances for another action may, though unlikely, to be the same (a tie). If the sum of the distances for an action and the sum of the distances for another action are the same, one of the two actions can be selected, for example, randomly. The process can be repeated for each read and then each position (or for each position and then each read), until all reads have the same sequence. With a low sequencing error rate (e.g., less than 1%), bases that are different from the “consensus base” at each position should be a very small fraction of bases at each position.

FIGS. 3A-3B show an example of generating a haplotype from multiple long reads. Scan from the beginning of each long read. If at a position, the base is not 100% the same amongst all reads, assume the base that appear the most amongst all reads as the “consensus base” or “truth base.” Then try to fix the reads that have different bases as the “consensus base.” Continue scanning and fixing the bases until reaching the end of each read. In the example shown in FIGS. 3A-3B, the three reads have the sequences ATCG, ATCT, and ATTCG. The most frequent base at the third position is cytosine (C). The third base (bolded and underlined) in read three, which is thymine (T), is different from the “consensus base.” The following three actions (or operations) can be performed independently on the third base in the read three: delete a base, add the “consensus base,” or change the base to the “consensus base.” If the third base is deleted, the distances (e.g., edit distances) between the modified read three with a sequence of ATCG and read one and read two are zero and zero, respectively. Thus the sum of the distances for the action of delete a base is zero. If the third base is changed to the “consensus base,” the modified read three has a sequence of ATCCG. The distances between the modified read three and read one and read two are one and one, respectively. Thus the sum of the distances for the action of change the base to the “consensus base” is two. If the “consensus base” is added or inserted into the third position of read three, the modified read three has a sequence of ATCTCG. The distances between the modified read three and read one and read two are two and two, respectively. Thus the sum of the distances for the action of add the “consensus” base is four. Because the resulting sum of distances is the smallest for the action of delete a base amongst the three actions, that action is selected. The sum of the distances for an action and the sum of the distances for another action may, though unlikely, to be the same (a tie). If the sum of the distances for an action and the sum of the distances for another action are the same, one of the two actions can be selected, for example, randomly. The modified read three is fixed at the third position. The process is repeated until reaching the end of all reads.

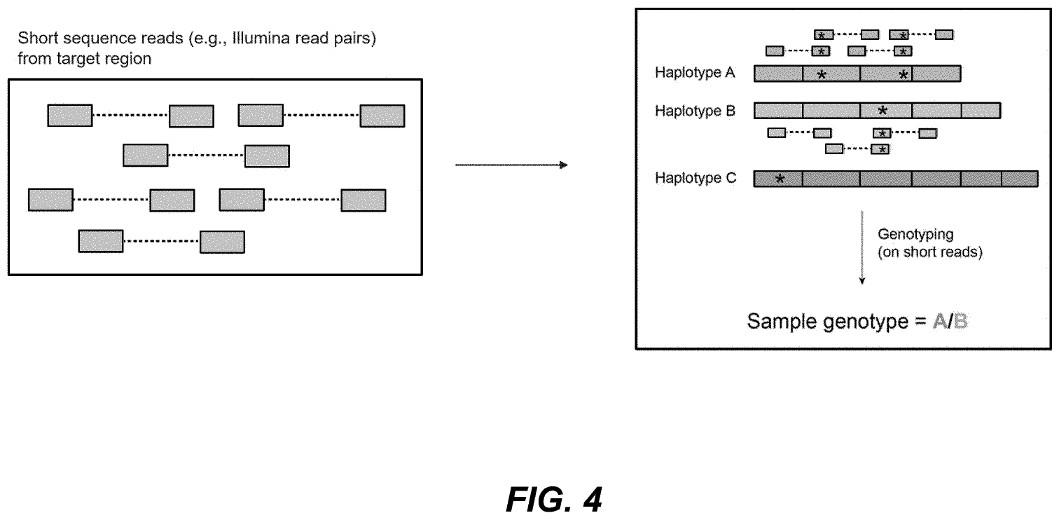

FIG. 4 shows an example of genotype VNTRs on short reads. Short reads (e.g., Illumina read pairs) can be extracted from the BAM file in the location of the VNTR. Each read can be realigned to each of the haplotypes in the VNTR haplotype database. In some embodiments, no gap is allowed in realignments. Each haplotype/read-pair combination can be scored. The VNTR genotyping model used in some embodiments for scoring is as the follows: For a read Rᵢ with L bases, its probability on the given haplotype H₁ is:

P(Rᵢ|H₁)=Πₖ₌₀^{L}P(Aₖ),

where Aₖ is the alignment on haplotype H₁ for the k^{th} base, and P(Aₖ) is pre-defined according to the match/mismatch status and the base quality score. On a read pair with fragment length Fᵢ, the above probability is expanded into:

P(Rᵢ₁,Rᵢ₂|H₁)=P(Rᵢ₁|H₁)P(Rᵢ₂|H₂)P(Fᵢ),

where P (Fᵢ) is estimated from the overall fragment length distribution in the given sample. Then probability of read Rᵢ for a specific diploid genotype G_{g}=H₁/H₂ is:

P(Rᵢ|G_{g}=H₁/H₂)=0.5*(P(Rᵢ|H₁)+P(Rᵢ|H₂)).

For each read, calculate P(Rᵢ|G_{g}) for every possible genotype.
The final genotype is derived from P(Rᵢ|G_{g}) of all N reads on all M possible genotypes, using the same Bayesian approach:

$$P ( {G}_{g} \vert {R}_{1} , … , {R}_{N} ) = \frac{P ( {G}_{g} ) {\prod }_{i = 0}^{N} P ( {R}_{i} \vert {G}_{g} )}{{\sum }_{g = 0}^{M} P ( {G}_{g} ) {\prod }_{i = 0}^{N} P ( {R}_{i} \vert {G}_{g} )}$$

The prior P(G_{g}) is estimated from the population frequency of G_{g}.

For some pure repeats without “star alleles”, more than one best genotype may be observed. Fragment length information may help to narrow down possible genotypes but cannot fully eliminate this ambiguity. In some embodiments, a confidence interval (CI) is reported as an estimate for VNTR lengths. The minimal set that can cover all these equally best genotypes can be first derived. Using this minimal set, CI can be reported as [shortest allele, longest allele] for each haplotype. For example, a VNTR is genotyped as having 2 possible genotypes with lengths: 50/60, 50/80 can be reported to have CI of [50,50] and [60,80].

VNTR genotyping accuracy. Improved VNTR genotyping accuracy was obtained using the genotyping method described herein (Table 1). Sixty samples were sequenced with PacBio HiFi. Illumina NovaSeq 6000 was used to test genotyping accuracy of VNTRs. A total number of 1,000 VNTRs were tested in this analysis. The genotyping method described herein had accuracies of 62%, 71%, and 78% measured by exact genotype, repeat length, and repeat length CI. Dragen v3.4 large variant detection accuracy, measured by the repeat length, was 16%. Paragraph (Chen, S., et al. Paragraph: a graph-based structural variant genotyper for short-read sequence data. Genome Biol 20, 291 (2019); the content of which is incorporated herein by reference in its entirety) large variant genotyping accuracy, measured by whether the repeat presence was correctly genotyped, was 38%.

TABLE 1
VNTR genotyping accuracy.

| Metric | Accuracy |
|---|---|
| Exact genotype | 0.62 |
| Repeat length | 0.71 |
| Repeat length CI | 0.78 |

“Star alleles” do not exist for some pure repeats. PacBio HiFi data may have low qualities in regions with large homopolymers. VNTR genotyping accuracy was improved (Table 2) by restricting the analysis to a whitelist that included assembled haplotypes with high qualities. Filtering criteria used to generate the whitelist included homopolymer length, purity of the repeat units, haplotype assembly quality, and repeat variability in the population. The performance shown in Table 2 was based on a whitelist that covered 60% of VNTRs originally tested. The improvements shown are relative to the performance of the VNTRs originally tested shown in Table 1. On the white list the VNTR genotyping performance was improved.

TABLE 2
Improved VNTR genotyping accuracy.

| | Accuracy-whitelist |
|---|---|
| Metric | (improvements) |
| Exact genotype | 0.69 (+7%) |
| Repeat length | 0.77 (+6%) |
| Repeat length CI | 0.83 (+5%) |

### Determining VNTR Status

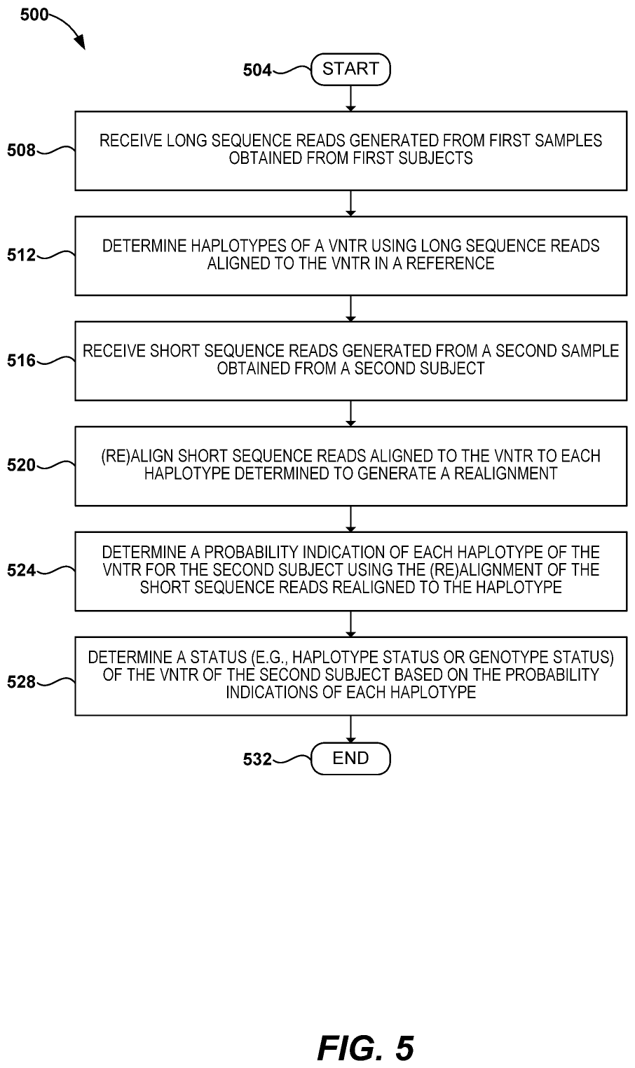

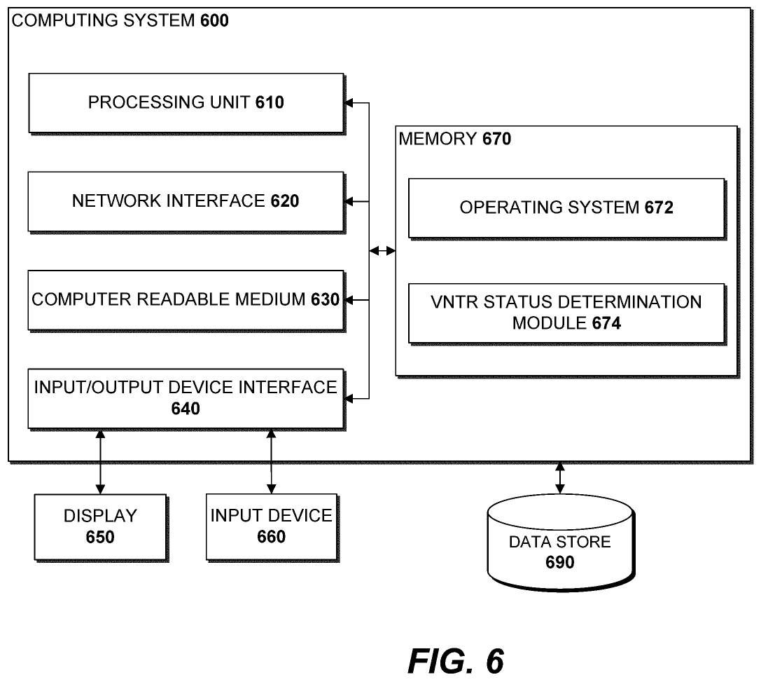

FIG. 5 is a flow diagram showing an exemplary method 500 of determining a VNTR status (e.g., VNTR haplotype or genotype), such as genotyping a VNTR. The method 500 may be embodied in a set of executable program instructions stored on a computer-readable medium, such as one or more disk drives, of a computing system. For example, the computing system 600 shown in FIG. 6 and described in greater detail below can execute a set of executable program instructions to implement the method 500. When the method 500 is initiated, the executable program instructions can be loaded into memory, such as RAM, and executed by one or more processors of the computing system 600. Although the method 500 is described with respect to the computing system 600 shown in FIG. 6, the description is illustrative only and is not intended to be limiting. In some embodiments, the method 500 or portions thereof may be performed serially or in parallel by multiple computing systems.

After the method 500 begins at block 504, the method 500 proceeds to block 508, where a computing system (such as the computing system 600 described with reference to FIG. 6) receives a plurality of long sequence reads generated from a plurality of first samples (or reference samples) obtained from a plurality of first subjects (or reference subjects). Long sequence reads are also referred to herein as long reads. Long sequence reads can be, for example, PacBio HiFi reads. A long sequence read can be, for example, 5 kilo base pairs (kbps), 6 kbps, 7 kbps, 8 kbps, 9 kbps, 10 kbps, 11 kbps, 12 kbps, 13 kbps, 14 kbps, 15 kbps, 20 kbps, 25 kbps, 30 kbps, or more. For example, the plurality of long sequence reads comprises sequence reads that are about 10 kbps to about 20 kbps. One, one or more, of each of the plurality of long sequence read (or long sequence reads that are aligned to the left flanking region and the right flanking region of the VNTR at block 512) can have a high accuracy, such as 95%, 96%, 97%, 98%, 99%, or more. The plurality of first samples can comprise at least, 40, 50, 60, 70, 80, 90, 100, 200, 300, 400, 500, 1000, or more samples. The plurality of long sequence reads can be generated by targeted sequencing or whole genome sequencing (WGS). The WGS can be clinical WGS (cWGS). The plurality of first subjects can comprises human subject.

The method 500 proceeds from block 508 to block 512, where the computing system determines a plurality of haplotypes of a VNTR (or a database of haplotypes of a VNTR) using long sequence reads, of the plurality of long sequence reads, aligned to the VNTR in a reference (See FIG. 2 and accompanying descriptions for an illustration). A reference can be, for example, a reference human genome sequence, such as hg19 or hg38. A haplotype of the plurality of haplotypes of the VNTR is associated with a disease. Non-limiting examples of the disease include bipolar disorder, MCKD1, stroke, CAD, FSHD, ADHD, Parkinson's, Diffuse panbronchiolitis (DPB), monogenic diabetes, T1D; T2D; Obesity, OCD, ADHD, osteochondritis dissecans, Kawasaki, ATF in stroke, BPSD, Alzheimer's, OCD, anxiety, schizophrenia, metastatic colorectal cancer, Kawasaki, or progressive myoclonic epilepsy 1A. The VNTR can be present in the coding region or non-coding region. The VNTR can be present in the 5′ untranslated region (UTR), promoter, intron, or 3′ UTR. The gene that includes, or is affected by, the VNTR can be, for example, PER3, MUC1, IL1RN, DUX4, DAT1, MUC21, CEL, INS, DRD4, ACAN, ZFHX3, GP1BA, SERT, SERT, HIC1, MMP9, CSTB, or MAOA.

Each haplotype of the plurality of haplotypes of the VNTR can comprise a plurality of copies of a repeat unit. The repeat unit can be (or be at least or be more than) 6 bps, 7 bps, 8 bps, 9 bps, 10 bps, 11 bps, 12 bps, 13 bps, 14 bps, 15 bps, 16 bps, 17 bps, 18 bps, 19 bps, 20 bps, 30 bps, 40 bps, 50 bps, 60 bps, 70 bps, 80 bps, 90 bps, 100 bps, 150 bps, 200 bps, or more in length. The number of the plurality of copies can be (or be at least or be more than) 2, 3, 4, 5, 6, 7, 8, 9, 10, 11, 12, 13, 14, 15, 16, 17, 18, 19, 20, 30, 40, 50, 60, 70, 80, 90, 100, 150, 200, 300, 400, 500, or more. The pathogenic copy number can be equal to, more than, or less than, the copy number in the reference.

Two copies of the repeat unit of a haplotype can include differentiating bases at certain positions (referred to herein as differentiating positions). For example, sequences of two copies of the plurality of copies of the repeat unit of a haplotype of the plurality of haplotypes are different at one or more differentiating positions (e.g., 2, 3, 4, 5, 10, 20, or more, positions). A star allele of a haplotype can include differentiating bases at these positions. A star allele can include positions that can help to distinguish two or more haplotypes from each other. The sequences of the two copies of the plurality of copies of the repeat unit of a haplotype have (or have at least) 70%, 75%, 80%, 85%, 90%, 95%, 99%, or more, sequence identity. Sequences of two copies of the plurality of copies of the repeat unit of a haplotype of the plurality of haplotypes can be identical. In some embodiments, two haplotypes of the plurality of haplotypes of the VNTR comprise different numbers of copies of the repeat unit.

A copy of the repeat unit in each of two haplotypes can include differentiating bases at certain positions (referred to herein as differentiating positions). For example, two haplotypes of the plurality of haplotypes of the VNTR comprise an identical number of copies of the repeat unit. A sequence of a copy of the repeat unit of one of the two haplotypes and a sequence of a copy of the repeat unit of the other one of the two haplotypes can be different at one or more differentiating positions. A star allele of a haplotype can include differentiating bases at these positions. The sequences of the two copies can have (or have at least) 70%, 75%, 80%, 85%, 90%, 95%, 99%, or more, sequence identity. A sequence of a copy of the repeat unit of one of the two haplotypes and a sequence of a copy of the repeat unit of the other one of the two haplotypes can be identical.

To determine the plurality of haplotypes of the VNTR, the computing system can build or create a database comprising the plurality of haplotypes of the VNTR. To determine the plurality of haplotypes of the VNTR, the computing system can for each of the plurality of first samples: extract the long sequence reads of the plurality of long sequence reads of the first sample aligned to the VNTR in the reference. The computing system can realign the long sequence reads extracted to a left flanking region and a right flanking region of the VNTR to determine aligned long sequence reads. Aligned long sequence reads can be long sequence reads that are aligned with the left flanking region and the right flanking region. Aligned long sequence reads can be long sequence reads with associated alignments e.g., relative to the left flanking region and the right flanking region. The computing system can determine a haplotype of the plurality of haplotypes based on the aligned long sequence reads each with an alignment score above an alignment threshold (e.g., 80%, 85%, 90%, 95%, 99%, or 100%, sequence identity). The alignment threshold can be predetermined. In some embodiments, the alignment threshold is determined using a number of samples, such as 100, 200, 300, 400, 500, 1000, 2000, 3000, 4000, 5000, or more or less, samples. At least one of the long sequence reads of the plurality of long sequence reads of the first sample can be aligned to the VNTR. At least one of the long sequence reads of the plurality of long sequence reads of the first sample can be realigned to the left flanking region and the right flanking region span the VNTR. In some embodiments, to determine the haplotype of the plurality of haplotypes of the VNTR, the computing system can trim sequences, of the aligned long sequence reads each with the alignment score above the alignment threshold, aligned to the left flanking region and the right flanking region to generate trimmed long sequence reads. The computing system can determine the haplotype of the plurality of haplotypes based on the trimmed long sequence reads.

The first sample can be heterozygous for the VNTR. The plurality of trimmed long sequence reads can be clustered into two clusters. To determine the haplotype of the plurality of haplotypes, the computing system can determine two haplotypes of the plurality of haplotypes of the VNTR based on the trimmed long sequence reads. To determine the two haplotypes, the computing system can cluster the trimmed long sequence reads into two clusters. The computing system can cluster the trimmed long sequence reads into two clusters based on lengths of the trimmed long sequence reads. The computing system can cluster the trimmed long sequence reads into two clusters using a clustering method. The clustering method can comprise k-means clustering (e.g., with k equals 2). The clustering method can comprise hierarchical clustering. The clustering method can be performed using, for example, a connectivity model, a centroid model, a distribution model, or a density model. The computing system can determine a first haplotype of the two haplotypes based on the trimmed long sequence reads in a first cluster of the two clusters. The computing system can determine a second haplotype of the two haplotypes based on the trimmed long sequence reads in a second cluster of the two clusters. In some embodiments, the trimmed long sequence reads comprise a first plurality of trimmed long sequence reads and a second plurality of trimmed long sequence reads with different lengths. A cluster can have a length (e.g., the average length of trimmed long sequence reads in the cluster) of about 1 kilo base pairs (kbps) 2 kbps, 3 kbps, 4 kbps, 5 kbps, 10 kbps, 15 kbps, 20 kbps, 30 kbps, 40 kbps, 50 kbps, 100 kbps, or more. The lengths of the two clusters (e.g., the average length of trimmed long sequence reads in each cluster) can differ by about, or by at least, 1 kbps 2 kbps, 3 kbps, 4 kbps, 5 kbps, 10 kbps, 15 kbps, 20 kbps, 30 kbps, 40 kbps, 50 kbps, 100 kbps, or more. For example, one cluster is about 5 kbps in length, and the other cluster is about 30 in length, and the lengths of the two clusters can differ by about 25 kbps. The first cluster can comprise all, substantially all (e.g., 90%, 95%, 99%, or more), or a majority (e.g., 51%, 60%, 70%, 80%, or more) of the first plurality of trimmed long sequence reads. The second cluster can comprise all, substantially all, or majority of the second plurality of trimmed long sequence reads.

The first sample can be homozygous for the VNTR. The first sample can be heterozygous for the VNTR. The plurality of trimmed long sequence reads cannot be clustered into two clusters. To determine the haplotype of the plurality of haplotypes, the computing system can determine only one haplotype of the plurality of haplotypes based on the trimmed long sequence reads. To determine the only one haplotype, the computing system can cluster the trimmed long sequence reads into only one cluster. For example, the separation between the trimmed long sequence reads can be sufficiently small such that the trimmed long sequence reads are not clustered into two clusters and/or are clustered into only one cluster. The computing system can cluster the trimmed long sequence reads into only one cluster based on lengths of the trimmed long sequence reads. The computing system can cluster the trimmed long sequence reads into only one cluster using a clustering method. The clustering method can comprise k-means clustering (e.g., with k equals 2). For example, the difference between the lengths of the trimmed long sequence reads can be sufficiently small such that the trimmed long sequence reads are not clustered into two clusters and/or are clustered into only one cluster using k-means clustering with k equals 2. The clustering method can comprise hierarchical clustering. The clustering method can be performed using, for example, a connectivity model, a centroid model, a distribution model, or a density model. The computing system can determine the only one haplotype based on the trimmed long sequence reads.

To determine the haplotype of the plurality of haplotypes of the VNTR, the computing system can determine a consensus sequence of the trimmed long sequence reads (see FIG. 2 and accompanying descriptions for an illustration). In some embodiments, to determine the consensus sequence of the trimmed long sequence reads, the computing system can perform the following for each position of each of the trimmed long sequence reads with a base that is not the most frequent base amongst the trimmed long sequence reads at the position (traversing through a (corresponding) position for all of the trimmed long sequence reads before proceeding to the next position for all of the trimmed long sequence reads, or traversing through each position of a trimmed long sequence read before traversing through each position of another trimmed long sequence read). The computing system can modify the trimmed long sequence read at the position using each of a plurality of operations (e.g., a delete operation, an insert operation, and a replace operation) independently and determine a sum of distances (e.g., edit distances) between (i) a modified trimmed long sequence read resulting from the operation on the trimmed long sequence read at the base and (ii) the trimmed long sequence reads other than the trimmed long sequence read being modified. The computing system can modify the trimmed long sequence at the base using the operation of the plurality of operations resulting in the smallest sum of distances (e.g., edit distances) amongst the plurality of operations. Alternatively, the computing system can replace the trimmed long sequence read with the modified trimmed long sequence read corresponding to the smallest sum of distances (e.g., edit distances). In some embodiments, the plurality of operations comprises deleting the base of the trimmed long sequence at the position. The plurality of operations can comprise inserting the most frequent base at the position into the trimmed long sequence at the position. The plurality of operations can comprise replacing the base of the trimmed long sequence at the position with the most frequent base at the position.

In some embodiments, to determine the consensus sequence of the trimmed long sequence reads, the computing system can, perform the following for each position of the trimmed long sequence reads (traversing through a (corresponding) position for all of the trimmed long sequence reads before proceeding to the next position for all of the trimmed long sequence reads, or traversing through each position of a trimmed long sequence read before traversing through each position of another trimmed long sequence read). The computing system can determine a most frequent base amongst bases of the trimmed long sequence reads at the position. For each of the trimmed long sequence reads with bases at the position that are not the most frequent base at the position, the computing system can determine a sum of distances (e.g., edit distances) between (i) a modified trimmed long sequence read resulting from each of a plurality of operations on the trimmed long sequence read and (ii) the trimmed long sequence reads other than the trimmed long sequence read being modified. The computing system can determine the smallest sum of distances (e.g., edit distances) amongst the sums of distances (e.g., edit distances). The computing system can modify the trimmed long sequence read at the base with the operation resulting in the smallest sum of distances (e.g., edit distances). Alternatively, the computing system can replace the trimmed long sequence read with the modified trimmed long sequence read corresponding to the smallest sum of distances (e.g., edit distances).

In some embodiments, long sequence reads and/or haplotypes can be filtered out or discarded based on quality criteria such as sequencing quality, homopolymer length, purity of the repeat units, haplotype assembly quality, and repeat variability in the population. Qualities of long sequence reads of the plurality of long sequence reads (before or after the long sequence reads are aligned to the VNTR in the reference) can satisfy one or more quality criteria. Long sequence reads that do not satisfy one or more quality criteria (or filtering criteria) can be filtered out or discarded from used to determine the haplotype of the plurality of haplotypes. Quality criteria can include sequencing quality (e.g., base call accuracy, such as Phred quality score) and homopolymer length. For example, long sequence reads can have low qualities in regions with large homopolymers. Such low-quality long sequence reads can be discarded from determining the haplotype of the plurality of haplotypes. In some embodiments, qualities of the plurality of haplotypes satisfy one or more quality criteria (or filtering criteria). A haplotype that does not satisfy one or more quality criteria can be filtered out or discarded. The quality criteria can include, for example, homopolymer length, purity of the repeat units, haplotype assembly quality, and/or repeat variability in the population. The remaining haplotypes can be a whitelist of haplotypes. The whitelist of haplotypes can be used at one or more subsequent blocks of the method 500. The whitelist of haplotypes can include, for example, about 50%, 60%, 70%, or 80%, of all the haplotypes first determined (including both the whitelist of haplotypes and haplotypes that have been filtered out or discarded). The plurality of haplotypes can include the whitelist of haplotypes, not the haplotypes that have been filtered out.

In some embodiments, instead of receiving a plurality of long sequence reads at block 508 and determining a plurality of haplotypes of a VNTR using the plurality of long sequence reads at block 512, the computing system receives a plurality of haplotypes of a VNTR (or a database of haplotypes of a VNTR). Alternatively or additionally, a plurality of haplotypes of a VNTR (or a database of haplotypes of a VNTR) is stored in the memory of the computing system. The plurality of haplotypes can be determined using long sequence reads, of a plurality of long sequence reads, aligned to the VNTR in a reference as describe with reference to block 512.

The method 500 proceeds from block 512 to block 516, where the computing system receives a plurality of short sequence reads generated from a second sample (or a test sample) obtained from a second subject. Short sequence reads are also referred to herein as short reads. Short sequence reads can be, for example, 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more base pairs (bps) in length each. For example, short sequence reads are about 100 bps to about 1000 bps in length each. The short sequence reads can comprise paired-end sequence reads. The sequence reads can comprise single-end sequence reads. The short sequence reads can be generated by targeted sequencing. The short sequence reads can be generated by whole genome sequencing (WGS). The short sequence reads can be generated by whole genome sequencing (WGS). The WGS can be clinical WGS (cWGS). A second sample can comprise cells, cell-free DNA, cell-free fetal DNA, amniotic fluid, a blood sample, a biopsy sample, or a combination thereof. The second subject can be a human subject. In some embodiments, the plurality of first subjects comprises the second subject. In some embodiments, the plurality of first samples comprises the second sample.

The computing system can store the sequence reads in memory. The computing system can load sequence reads into memory. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, Calif.).

The method 500 proceeds from block 516 to block 520, where the computing system, for each of the plurality of haplotypes of the VNTR, (re)aligns short sequence reads, of the plurality of short sequence reads aligned to the VNTR, to the haplotype to generate a realignment. In some embodiments, no gap is allowed in realignments. In some embodiments, gaps are allowed in realignments. The computing system can (re)align short sequence reads to the haplotype using an aligner or an alignment method such as Burrows-Wheeler Aligner (BWA), ISAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRiMP, SLIDER, SOAP, SOAP2, SOAP3 and SOAP3-dp, SOCS, SSAHA and SSAHA2, Stampy, SToRM, Subread and Subjunc, Taipan, UGENE, VelociMapper, XpressAlign, and ZOOM.

The method 500 proceeds from block 520 to block 524, where the computing system determines a probability indication of each of the plurality of haplotypes of the VNTR for the second subject using the realignment of the short sequence reads (re)aligned to the haplotype. The computing system can determine two or more haplotypes of the plurality of haplotypes with each haplotype having the probability indication that satisfies a probability criterium. The probability indication of each of the plurality of haplotypes of the VNTR comprises a probability of each of the plurality of haplotypes of the VNTR. The probability criterium can comprise a probability threshold (e.g., 50%, 55%, 60%, 65%, 70%, 75%, 80%, 85%, 90%, or 95%). The probability threshold can be predetermined. In some embodiments, the probability threshold is determined using a number of samples, such as 100, 200, 300, 400, 500, 1000, 2000, 3000, 4000, 5000, or more or less, samples. The probability criterium can comprise the highest probability (or highest few probabilities, such as highest 2, 3, 4, 5, or more probabilities) amongst each of the plurality of haplotypes.

Alternatively or additionally, the computing system determines a probability indication of each pair of haplotypes of the plurality of haplotypes of the VNTR for the second subject using the realignment of the short sequence reads (re)aligned to the haplotype. The computing system can determine one or more pairs of haplotypes of the plurality of haplotypes with each pair having the probability indication that satisfies a probability criterium. The probability indication of each pair of haplotypes of the plurality of haplotypes of the VNTR can comprise a probability of each pair of haplotypes of the VNTR. The probability criterium can comprise a probability threshold (e.g., 50%, 55%, 60%, 65%, 70%, 75%, 80%, 85%, 90%, or 95%). The probability criterium can comprise the highest probability (or highest few probabilities, such as highest 2, 3, 4, 5, or more probabilities) amongst each pair of haplotypes of the plurality of haplotypes.

For example, a score (e.g., a probability indication) of each haplotype/sequence read combination can be determined. The VNTR genotyping model used in some embodiments for scoring is as the follows:

For a read Rᵢ with L bases, its probability on the given haplotype H₁ is:

P(Rᵢ|H₁)=Πₖ₌₀^{L}P(Aₖ),

where Aₖ is the alignment on haplotype H₁ for the k^{th} base, and
P(Aₖ) can be predefined according to the match/mismatch status and the base quality score.
On a read pair with fragment length Fᵢ, the above probability can be expanded into:

P(Rᵢ₁,Rᵢ₂|H₁)=P(Rᵢ₁|H₁)P(Rᵢ₂|H₂)P(Fᵢ),

where P (Fᵢ) is estimated from the overall fragment length distribution in the given sample. Then probability of read Rᵢ for a specific diploid genotype G_{g}=H₁/H₂ is:

P(Rᵢ|G_{g}=H₁/H₂)=0.5*(P(Rᵢ|H₁)+P(Rᵢ|H₂)).

For each read, calculate P(Rᵢ|G_{g}) for every possible genotype.
The final genotype can be derived from P(Rᵢ|G_{g}) of all N reads on all M possible genotypes, using the same Bayesian approach:

$$P ( {G}_{g} \vert {R}_{1} , … , {R}_{N} ) = \frac{P ( {G}_{g} ) {\prod }_{i = 0}^{N} P ( {R}_{i} \vert {G}_{g} )}{{\sum }_{g = 0}^{M} P ( {G}_{g} ) {\prod }_{i = 0}^{N} P ( {R}_{i} \vert {G}_{g} )}$$

The prior P(G_{g}) can be estimated from the population frequency of G_{g}.

The method 500 proceeds from block 524 to block 528, where the computing system determines a status of the VNTR of the second subject based on the probability indications of each of the plurality of haplotypes. An accuracy of the status of the VNTR can be at least 50%, 55%, 60%, 65%, 70%, 75%, 80%, 85%, 90%, or 95%. The status of the VNTR can comprise a haplotype status of the VNTR. The haplotype status can comprise a haplotype, a length of the haplotype, and/or a confidence interval (CI) of the length of the haplotype. The confidence interval can comprise a shortest length of the haplotype and a longest length of the haplotype.

The status of the VNTR can comprise a genotype status of the VNTR. The genotype status can comprise a genotype, lengths of the haplotypes of the genotype, and/or a confidence interval of the length of each of the haplotypes of the genotype. The confidence interval can comprise a shortest length of each of the haplotypes and a longest length of each of the haplotypes. The computing system can determine lengths of the two or more haplotypes determined. The shortest length of the haplotype can be the shortest length of the lengths of the two or more haplotypes determined. The longest length of the haplotypes can be the longest length of the lengths of the two or more haplotypes determined.

In some embodiments, the computing systems generates a user interface (UI), such as a graphical user interface, comprising or representing the status of the VNTR. The UI can include, for example, a dashboard. The UI can include one or more UI elements. A UI element can comprise or represent the status of the VNTR. A UI element can be a window (e.g., a container window, browser window, text terminal, child window, or message window), a menu (e.g., a menu bar, context menu, or menu extra), an icon, or a tab. A UI element can be for input control (e.g., a checkbox, radio button, dropdown list, list box, button, toggle, text field, or date field). A UI element can be navigational (e.g., a breadcrumb, slider, search field, pagination, slider, tag, icon). A UI element can informational (e.g., a tooltip, icon, progress bar, notification, message box, or modal window). A UI element can be a container (e.g., an accordion).

The method 500 ends at block 532.

### Execution Environment

FIG. 6 depicts a general architecture of an example computing device 600 configured for determining a VNTR status, such as genotyping a VNTR. The general architecture of the computing device 600 depicted in FIG. 6 includes an arrangement of computer hardware and software components. The computing device 600 may include many more (or fewer) elements than those shown in FIG. 6. It is not necessary, however, that all of these generally conventional elements be shown in order to provide an enabling disclosure. As illustrated, the computing device 600 includes a processing unit 610, a network interface 620, a computer readable medium drive 630, an input/output device interface 640, a display 650, and an input device 660, all of which may communicate with one another by way of a communication bus. The network interface 620 may provide connectivity to one or more networks or computing systems. The processing unit 610 may thus receive information and instructions from other computing systems or services via a network. The processing unit 610 may also communicate to and from memory 670 and further provide output information for an optional display 650 via the input/output device interface 640. The input/output device interface 640 may also accept input from the optional input device 660, such as a keyboard, mouse, digital pen, microphone, touch screen, gesture recognition system, voice recognition system, gamepad, accelerometer, gyroscope, or other input device.

The memory 670 may contain computer program instructions (grouped as modules or components in some embodiments) that the processing unit 610 executes in order to implement one or more embodiments. The memory 670 generally includes RAM, ROM and/or other persistent, auxiliary or non-transitory computer-readable media. The memory 670 may store an operating system 672 that provides computer program instructions for use by the processing unit 610 in the general administration and operation of the computing device 600. The memory 670 may further include computer program instructions and other information for implementing aspects of the present disclosure.

For example, in one embodiment, the memory 670 includes a VNTR status determination module 674 for determining a VNTR status, such as the method 500 described with reference to FIG. 5. In addition, memory 670 may include or communicate with the data store 690 and/or one or more other data stores that store one or more inputs, one or more outputs, and/or one or more results (including intermediate results) of determining a VNTR status of the present disclosure, such the long reads, the plurality of haplotypes determined, the short reads, and the VNTR status (e.g., haplotypes or genotype of a sample) determined.

### ADDITIONAL CONSIDERATIONS

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

1.-41. (canceled)

42. A system for determining a variable number tandem repeat (VNTR) status comprising:
non-transitory memory configured to store executable instructions and a plurality of haplotypes of a VNTR; and
a hardware processor in communication with the non-transitory memory, the hardware processor programmed by the executable instructions to perform:
receiving a plurality of short sequence reads generated from a test sample obtained from a test subject;
for each of the plurality of haplotypes of the VNTR, realigning short sequence reads, of the plurality of short sequence reads aligned to the VNTR, to the haplotype to generate a realignment;
determining a probability of each of the plurality of haplotypes for the test subject using the realignment of the short sequence reads realigned to the haplotype; and
determining a status of the VNTR of the test subject.

43. The system of claim 42, wherein the plurality of haplotypes of the VNTR is determined using long sequence reads of a plurality of long sequence reads aligned to the VNTR in a reference, and wherein the plurality of long sequence reads is generated from a plurality of reference samples obtained from a plurality of reference subjects.

44. The system of claim 43, wherein the plurality of haplotypes of the VNTR is determined by:
for each of the plurality of samples:
extracting the long sequence reads of the plurality of long sequence reads of the test sample aligned to the VNTR in the reference;
realigning the long sequence reads extracted to a left flanking region and a right flanking region of the VNTR to determine aligned long sequence reads; and
determining a haplotype of the plurality of haplotypes based on the aligned long sequence reads each with an alignment score above an alignment threshold.

45. (canceled)

46. The system of claim 44, wherein the haplotype of the plurality of haplotypes of the VNTR is determined by:
trimming sequences, of the aligned long sequence reads each with the alignment score above the alignment threshold, aligned to the left flanking region and the right flanking region to generate trimmed long sequence reads; and
determining the haplotype of the plurality of haplotypes based on the trimmed long sequence reads.

47. The system of claim 44, wherein the reference sample is homozygous for the VNTR, and the haplotype of the plurality of haplotypes of the VNTR is determined to comprise only one haplotype of the plurality of haplotypes based on the trimmed long sequence reads.

48. (canceled)

49. The system of claim 44, wherein the reference sample is heterozygous for the VNTR and wherein the haplotype of the plurality of haplotypes of the VNTR is determined to comprise two haplotypes of the plurality of haplotypes based on the trimmed long sequence reads.

50. (canceled)

51. (canceled)

52. The system of claim 44, the haplotype of the plurality of haplotypes of the VNTR is determined by: determining a consensus sequence of the trimmed long sequence reads.

53.-55. (canceled)

56. The system of claim 43, wherein qualities of the long sequence reads of the plurality of long sequence reads aligned to the VNTR in the reference and/or qualities of the plurality of haplotypes satisfy quality criteria.

57. The system of claim 42, wherein the status of the VNTR comprises a haplotype status of the VNTR and/or a genotype status of the VNTR, optionally wherein the haplotype status comprises a haplotype, a length of the haplotype, and a confidence interval of the length of the haplotype, and optionally wherein the genotype status comprises a genotype, lengths of the haplotypes of the genotypes, and a confidence interval of the length of each of the haplotypes of the genotype.

58. (canceled)

59. (canceled)

60. The system of claim 42, wherein the probability indication of each of the plurality of haplotypes of the VNTR comprises a probability of each of the plurality of haplotypes of the VNTR, and wherein the probability criterium comprises a probability threshold.

61. The system of claim 42, wherein an accuracy of the haplotype status is at least 60%.

62. The system of claim 42, wherein the plurality of long sequence reads comprises sequence reads that are about 10,000 base pairs to about 20,000 base pairs in length each.

63. The system of claim 42, wherein the plurality of short sequence reads comprises sequence reads that are about 100 base pairs to about 1000 base pairs in length each.

64.-70. (canceled)

71. The system of claim 42, wherein each haplotype of the plurality of haplotypes of the VNTR comprises a plurality of copies of a repeat unit.

72. The system of claim 71, wherein the repeat unit is more than six base pairs in length.

73. The system of claim 71, wherein the number of the plurality of copies is at least three.

74. The system of claim 71, wherein sequences of two copies of the plurality of copies of the repeat unit of a haplotype of the plurality of haplotypes are different at one or more differentiating positions.

75.-80. (canceled)

81. The system of claim 42, wherein a haplotype of the plurality of haplotypes of the VNTR is associated with a disease.

82. The system of claim 42, wherein the hardware processor is programmed by the executable instructions to perform: generating a user interface (UI) comprising a UI element representing the status of the VNTR.

83. A method for determining a variable number tandem repeat (VNTR) status comprising:
under control of a hardware processor:
receiving a plurality of long sequence reads generated from a plurality of first samples obtained from a plurality of first subjects;
determining a plurality of haplotypes of a VNTR using long sequence reads of the plurality of long sequence reads aligned to the VNTR in a reference;
receiving a plurality of short sequence reads generated from a second sample obtained from a second subject;
for each of the plurality of haplotypes of the VNTR, realigning short sequence reads, of the plurality of short sequence reads aligned to the VNTR, to the haplotype to generate a realignment;
determining a probability indication of each of the plurality of haplotypes of the VNTR for the second subject using the realignment of the short sequence reads realigned to the haplotype; and
determining a status of the VNTR of the second subject based on the probability indications of each of the plurality of haplotypes.


SEQUENCE LISTING (1 sequence)

SEQ ID NO: 1 - DNA, 48 bp, Homo sapiens
accccgagctagggtgcagcccggccgcactgcaggagacccaccagg